(19) United States
(12) Patent Application Publication
(10) Pub. No.: US 2012/0331579 A1
(43) Pub. Date: Dec. 27, 2012

(54) TRANSGENIC PLANT MALE STERILITY

(75) Inventors: Roger Parish, Warrandyte (AU); Song Feng Li, Bulleen (AU)

(73) Assignee: La TROBE UNIVERSITY, Bundoora (AU)

(21) Appl. No.: 13/509,265

(22) PCT Filed: Nov. 11, 2010
(86) PCT No.: PCT/AU10/01503

§ 371 Date: May 10, 2012

(30) Foreign Application Priority Data
Nov. 11, 2009 (AU) 2009905527

(51) Int. Cl. C12N 15/82; A01H 5/00; C12N 1/21; C12N 15/29; C07H 21/00; A01H 1/00; A01H 5/10

(52) U.S. Cl. 800/278; 435/320.1; 800/303; 800/298; 536/23.6; 536/23.1; 435/252.33; 435/252.2

(57) ABSTRACT

The invention relates to a reversible transgenic plant male sterility system wherein the male sterility is induced by amiRNA and a reversible transgenic plant male sterility system comprising a male sterility construct comprising an isolated nucleic acid encoding a precursor amiRNA encoding an amiRNA targeted to a gene involved in pollen development, and a male fertility restorer construct comprising an isolated nucleic acid encoding a mutated copy of the gene involved in pollen development, or multiple copies of said gene involved in pollen development, or a single copy of said gene involved in pollen development under the control of a strong promoter.

The invention relates to a transgenic plant male sterility system, using amiRNA (artificial microRNA) technology.

### BACKGROUND

Modern farming practices rely on the production of hybrid seed to maintain the heterosis of crop plants. To achieve hybrid seed production, it is imperative that self fertilisation be prevented. Pollination control mechanisms that have been developed to date include mechanical, chemical and genetic mechanisms.

The drawback of mechanical mechanisms of pollination control is that they are labour intensive, while chemical means of pollen control often require repeated applications of expensive and potentially environmentally undesirable chemicals.

Genetic systems involving cytoplasmic or nuclear genes may be used to generate male sterility. Cytoplasmic male sterility (CMS) is at present the most widely used mechanism of pollen control in crops. However, CMS has a number of disadvantages including increased disease susceptibility, breakdown of sterility under certain conditions, undesirable characteristics linked to restorer genes, unreliable restoration, etc. Nuclear-encoded male sterility (NMS) is caused by mutations in the nuclear genome to disrupt particular genes involved in plant fertility, thereby preventing functional proteins being produced from the nuclear genome.

An aim of the present invention is to develop a further reversible transgenic plant male sterility system.

### SUMMARY

In a first aspect, the invention provides a reversible transgenic plant male sterility system wherein the male sterility is induced by amiRNA.

In a second aspect, the invention provides the use of amiRNA in a reversible transgenic plant male sterility system.

In a third aspect, the invention provides a reversible transgenic plant male sterility system comprising a male sterility construct, said male sterility construct comprising an isolated nucleic acid encoding a precursor amiRNA encoding an amiRNA targeted to a gene involved in pollen development; and a male fertility restorer construct, said male fertility restorer construct comprising an isolated nucleic acid encoding a mutated copy of said gene involved in pollen development, said mutated copy comprising a mutation conferring resistance to said amiRNA targeted to said gene involved in pollen development.

In a fourth aspect the invention provides a method of producing a male sterile transgenic plant, said method comprising transforming a plant with an isolated nucleic acid encoding a precursor amiRNA targeted to a gene involved in pollen development.

In a fifth aspect the invention provides a method of producing a male fertility restorer transgenic plant capable of restoring fertility to the progeny of a male sterile plant produced according to the fourth aspect, said method comprising transforming a plant with an isolated nucleic acid encoding a mutated copy of a gene involved in pollen development, said mutated copy comprising a mutation conferring resistance to an amiRNA targeted to said gene involved in pollen development.

In a sixth aspect the invention provides a method of producing a male fertility restorer transgenic plant capable of restoring fertility to the progeny of a male sterile plant produced according to the fourth aspect, said method comprising transforming a plant with multiple copies of an isolated nucleic acid encoding a gene involved in pollen development, or a single copy of a gene involved in pollen development under a strong promoter, wherein the expression of the multiple copies of the gene involved in pollen development, or the copy of the gene involved in pollen development under the control of a strong promoter has the consequence that amiRNA down-regulation of the gene involved in pollen development in the male sterile plant produced according to the fourth aspect is overwhelmed and is no longer capable of inducing 100% male sterility.

In a seventh aspect the invention provides a male sterile transgenic plant produced by the method of the fourth aspect, and/or a male fertility restorer transgenic plant produced by the method of the fifth aspect or sixth aspect, as well as transgenic propagating material or progeny seed of such plants.

In an eighth aspect the invention provides a method of producing male fertile hybrid progeny from a male sterile plant produced by the method of the fourth aspect by fertilising the male sterile transgenic plant produced by the method of the fourth aspect with pollen from a male fertility restorer transgenic plant produced by the method of the fifth aspect or sixth aspect, and collecting the resulting male fertile hybrid seed.

In a ninth aspect the invention provides an isolated nucleic acid encoding a precursor amiRNA encoding an amiRNA targeted to a gene involved in pollen development.

In a tenth aspect the invention provides an isolated nucleic acid comprising a mutated copy of a gene involved in pollen development, said mutated copy comprising a mutation conferring resistance the amiRNA encoded by the nucleic acid of the eighth aspect.

The gene involved in pollen development contemplated by the invention includes, but is not limited to, a gene involved in anther development, pollen formation or pollen shedding, a member of the MYB class of transcription factors, a member of the R2-R3 family of the MYB class of transcription factors, MYB103, and Arabidopsis thaliana MYB103.

In an eleventh aspect the invention provides vectors, host cells and transgenic plants comprising the nucleic acid of the ninth or tenth aspects as well as transgenic seed, propagating material or progeny of such transgenic plants.

### BRIEF DESCRIPTION OF THE FIGURES

FIG. 1—A schematic representation of the amiRNA 1 construct.

FIG. 2—schematic representation of the preparation of the amiRNA 2 construct.

FIG. 3—A schematic representation of the amiRNA 1-2 construct.

FIG. 4—Pollen and silique morphology of wild-type and male sterile plants transgenic for the amiR103 precursor. Alexander's staining of wild-type pollen (a) and anther (c), amiR103 pollen (b) and anther (d), Black arrows indicating intact (c) and clumped (d) pollen grains. Wild-type elongated siliques (e) and aborted amiR103 siliques (f, white arrow).

FIG. 5—A schematic representation of the production of the Restorer 1 construct.

FIG. 6—A schematic representation of the production of the Restorer 2 construct.

FIG. 7—Hybrid seed production using artificial RNA targeting AtMYB103.

FIG. 8—Inducible male sterility and hybrid seed production using artificial miRNA.

### DETAILED DESCRIPTION

Complete plant male sterility is important for the effective production of hybrid seed, especially in species that have flowers comprising both male and female sexual organs. Existing methods of inducing plant male sterility are often susceptible to breakdown of sterility, or are too technically difficult to be attractive to farmers. The most promising method available before the present invention involved a repressor/restorer system in which the EAR motif was used to control male fertility. However, the effectiveness of the system in various crop plants has not been determined. The present invention using amiRNA provides an alternative reversible male sterility system for hybrid seed production, which may be more effective than the repressor/restorer system in some crop plants.

Another technique that has been investigated for the production of male sterile plants is antisense RNA. Antisense RNA, being an RNA based technology, is the closest relative of miRNA technology, but it fails to reliably produce 100% male sterility, and may result in other undesirable effects.

A relatively new technology that allows for manipulation and control of gene expression uses microRNA (miRNA) to down-regulate gene expression and/or to repress mRNA translation. miRNAs are small, endogenous non-coding RNAs which negatively regulate gene expression at the post-transcriptional level, through complementary binding of the miRNA to the target mRNA, and subsequent degradation of the target mRNA and/or the repression of the target mRNA translation. These miRNA molecules are 20 to 25 base, naturally occurring, single stranded RNA sequences that are assembled from precursor miRNA molecules transcribed from the genome. miRNAs have been shown to have essential regulatory roles in gene expression in both animals and plants. Plant miRNAs are known and generally exhibit high complementarity and have a small number of targets per molecule, while animal miRNAs usually affect hundreds of targets and exhibit limited complementarity.

Precursor miRNA molecules are post-transcriptionally processed into mature miRNA molecules. “Precursor miRNA” as used herein refers to the initial, approximately 80 to 250 base, mRNA transcript of the miRNA gene that is subsequently processed by a variety of endogenous enzymes, such as DICER-LIKE 1 (DCL1) and HYPONASTIC LEAVES 1 (HYL1) in Arabidopsis thaliana, into a 20 to 25 base, mature miRNA molecule with a hairpin conformation. The hairpin conformation of the mature miRNA molecule is achieved through complementary base pairing of the miRNA and miRNA-complementary regions within the mature miRNA molecule. This “mature miRNA” molecule is capable of binding to, and directing the degradation of, or interfering with the translation of, the mRNA transcribed from the gene to which it is targeted.

The inventors have investigated using amiRNA in a reversible plant male sterility system and have found, contrary to their expectations based on the inability of antisense technology to bring about 100% male sterility, that amiRNA can bring about 100% male sterility. Ten out of the 78 plant lines transgenic for the amiRNA103 precursor driven by the canola BnMYB103 promoter are completely male sterile (ie, all the plants in each of the male sterile lines are 100% male sterile).

The precursor miRNA backbone may be native to the plant being considered or it may be heterologous to the plant being considered or it may be a synthetic polynucleotide which does not occur in any plant species but retains the stem-loop structure recognised and processed by the plant processing complex, including DCL1.

An example of a precursor miRNA is miR319a from Arabidopsis thaliana (SEQ ID NOs: 1 to 3) which, once processed by DCL1, becomes a 21 base functional miRNA molecule. The miR319a does not naturally target the AtMYB103 transcription factor in A. thaliana.

An example of another miRNA precursor is miR159 (SEQ ID NOs: 4 to 6), which naturally targets AtMYB33 and AtMYB65. Over-expression of miR159 results in decreased levels of AtMYB33 and AtMYB65, abnormal flowers including abnormal anthers and delayed flowering time.

Other miRNA precursors derived from Arabidopsis thaliana contemplated for use in the invention are miRNA156a, miRNA156b, miRNA156c, miRNA156d, miRNA156e, miRNA156f, miRNA156g, miRNA156h, miRNA157a, miRNA157b, miRNA157c, miRNA157d, miRNA158a, miRNA158b, miRNA159a, miRNA159b, miRNA159c, miRNA160a, miRNA160b, miRNA160c, miRNA161a, miRNA162a, miRNA162b, miRNA163, miRNA164a, miRNA164b, miRNA164c, miRNA165a, miRNA165b, miRNA166a, miRNA166b, miRNA166c, miRNA166d, miRNA166e, miRNA166f, miRNA166g, miRNA167a, miRNA167b, miRNA167c, miRNA167d, miRNA168a, miRNA168b, miRNA169a, miRNA169b, miRNA169c, miRNA169d, miRNA169e, miRNA169f, miRNA169g, miRNA169h, miRNA169i, miRNA169j, miRNA169k, miRNA169l, miRNA169m, miRNA169n, miRNA170, miRNA171a, miRNA171b, miRNA171c, miRNA172a, miRNA172b, miRNA172c, miRNA172d, miRNA172e, miRNA173, miRNA319a, miRNA319b, miRNA319c, miRNA390a, miRNA390b, miRNA391, miRNA393a, miRNA393b, miRNA394a, miRNA394b, miRNA395a, miRNA395b, miRNA395c, miRNA395d, miRNA395e, miRNA395f, miRNA396a, miRNA396b, miRNA397a, miRNA397b, miRNA398a, miRNA398b, miRNA398c, miRNA399a, miRNA399b, miRNA399c, miRNA399d, miRNA399e, miRNA399f, miRNA400, miRNA401, miRNA402, miRNA403, miRNA404, miRNA405a, miRNA405b, miRNA405d, miRNA406, miRNA407, miRNA408, miRNA413, miRNA414, miRNA415, miRNA416, miRNA417, miRNA418, miRNA419, miRNA420, miRNA426, miRNA447a, miRNA447b, miRNA447c, miRNA472, miRNA771, miRNA773, miRNA773, miRNA774, miRNA775, miRNA776, miRNA777, miRNA778, miRNA779, miRNA780, miRNA781, miRNA782, miRNA783, miRNA822, miRNA823, miRNA824, miRNA825, miRNA826, miRNA827, miRNA828, miRNA829, miRNA830, miRNA831, miRNA832, miRNA833, miRNA834, miRNA835, miRNA836, miRNA837, miRNA838, miRNA839, miRNA840, miRNA841, miRNA842, miRNA843, miRNA844, miRNA845a, miRNA845b, miRNA846, miRNA847, miRNA848, miRNA849, miRNA850, miRNA851, miRNA852, miRNA853, miRNA854a, miRNA854b, miRNA854c, miRNA854d, miRNA855, miRNA856, miRNA857, miRNA858, miRNA859, miRNA860, miRNA861, miRNA862, miRNA863, miRNA864, miRNA865, miRNA866, miRNA867, miRNA868, miRNA869, miRNA870.

miRNA precursors derived from Oryza sativa contemplated for use in the invention are miRNA156a, miRNA156b, miRNA156c, miRNA156d, miRNA156e, miRNA156f, miRNA156g, miRNA156h, miRNA156i, miRNA156j, miRNA156k, miRNA156l, miRNA159a, miRNA159b, miRNA159c, miRNA159d, miRNA159e, miRNA159f, miRNA160a, miRNA160b, miRNA160c, miRNA160d, miRNA160e, miRNA160f, miRNA162a, miRNA162b, miRNA164a, miRNA164b, miRNA164c, miRNA164d, miRNA164e, miRNA164f, miRNA166a, miRNA166b, miRNA166c, miRNA166d, miRNA166e, miRNA166f, miRNA166g, miRNA166h, miRNA166i, miRNA166j, miRNA166k, miRNA166l, miRNA166m, miRNA166n, miRNA167a, miRNA167b, miRNA167c, miRNA167d, miRNA167e, miRNA167f, miRNA167g, miRNA167h, miRNA167i, miRNA167j, miRNA168a, miRNA168b, miRNA169a, miRNA169b, miRNA169c, miRNA169d, miRNA169e, miRNA169f, miRNA169g, miRNA169h, miRNA169i, miRNA169j, miRNA169k, miRNA169l, miRNA169m, miRNA169n, miRNA169o, miRNA169p, miRNA169q, miRNA171a, miRNA171b, miRNA171c, miRNA171d, miRNA171e, miRNA171f, miRNA171g, miRNA171h, miRNA171i, miRNA172a, miRNA172b, miRNA172c, miRNA172d, miRNA319a, miRNA319b, miRNA390, miRNA393, miRNA393b, miRNA394, miRNA395a, miRNA395b, miRNA395c, miRNA395d, miRNA395e, miRNA395f, miRNA395g, miRNA395h, miRNA395i, miRNA395j, miRNA395k, miRNA395l, miRNA395m, miRNA395n, miRNA395o, miRNA395p, miRNA395q, miRNA395r, miRNA395s, miRNA395t, miRNA395u, miRNA395v, miRNA395w, miRNA396a, miRNA396b, miRNA396c, miRNA396d, miRNA396e, miRNA397a, miRNA397b, miRNA398a, miRNA398b, miRNA399a, miRNA399b, miRNA399c, miRNA399d, miRNA399e, miRNA399f, miRNA399g, miRNA399h, miRNA399i, miRNA399j, miRNA399k, miRNA408, miRNA413, miRNA414, miRNA415, miRNA416, miRNA417, miRNA418, miRNA419, miRNA420, miRNA426, miRNA435, miRNA437, miRNA438, miRNA439a, miRNA439b, miRNA439c, miRNA439d, miRNA439e, miRNA439f, miRNA439g, miRNA439h, miRNA439i, miRNA439j, miRNA440, miRNA441a, miRNA441b, miRNA441c, miRNA442, miRNA443, miRNA444, miRNA445a, miRNA445b, miRNA445c, miRNA445d, miRNA445e, miRNA445f, miRNA445g, miRNA445h, miRNA445i, miRNA446, miRNA528, miRNA529, miRNA530, miRNA531, miRNA535, miRNA806a, miRNA806b, miRNA806c, miRNA806d, miRNA806e, miRNA806f, miRNA806g, miRNA806h, miRNA807a, miRNA807b, miRNA807c, miRNA808, miRNA809a, miRNA 809b, miRNA809c, miRNA809d, miRNA809e, miRNA809f, miRNA809g, miRNA 809h, miRNA810, miRNA811a, miRNA811b, miRNA811c, miRNA812a, miRNA812b, miRNA812c, miRNA812d, miRNA812e, miRNA813, miRNA814a, miRNA814b, miRNA814c, miRNA815a, miRNA815b, miRNA815c, miRNA816, miRNA817, miRNA818a, miRNA818b, miRNA818c, miRNA818d, miRNA818e, miRNA819a, miRNA819b, miRNA819c, miRNA819d, miRNA819e, miRNA819f, miRNA819g, miRNA819h, miRNA819i, miRNA819j, miRNA819k, miRNA820a, miRNA820b, miRNA820c, miRNA821a, miRNA821b, miRNA821c.

miRNA precursors derived from Zea mays contemplated for use in the invention are miRNA156a, miRNA156b, miRNA156c, miRNA156d, miRNA156e, miRNA156f, miRNA156g, miRNA156h, miRNA156i, miRNA156j, miRNA156k, miRNA159a, miRNA159b, miRNA159c, miRNA159d, miRNA160a, miRNA160b, miRNA160c, miRNA160d, miRNA160e, miRNA160f, miRNA162, miRNA164a, miRNA164b, miRNA164c, miRNA164d, miRNA166a, miRNA166b, miRNA166c, miRNA166d, miRNA166e, miRNA166f, miRNA166g, miRNA166h, miRNA166i, miRNA166j, miRNA166k, miRNA1661, miRNA166m, miRNA167a, miRNA167b, miRNA167c, miRNA167d, miRNA167e, miRNA167f, miRNA167g, miRNA167h, miRNA167i, miRNA168a, miRNA168b, miRNA169a, miRNA169b, miRNA169c, miRNA169d, miRNA169e, miRNA169f, miRNA169g, miRNA169h, miRNA169i, miRNA169j, miRNA169k, miRNA171a, miRNA171b, miRNA171c, miRNA171d, miRNA171e, miRNA171f, miRNA171g, miRNA171h, miRNA171i, miRNA171j, miRNA171k, miRNA172a, miRNA172b, miRNA172c or miRNA172d, miRNA172e, miRNA319a, miRNA319b, miRNA319c, miRNA319d, miRNA393, miRNA394a, miRNA394b, miRNA395a, miRNA395b, miRNA395c, miRNA396a, mIRNA396b, miRNA399a, miRNA399b, miRNA399c, miRNA399d, miRNA399e, miRNA399f, miRNA408.

miRNA precursors derived from soy contemplated for use in the invention are miRNA156a, miRNA156b, miRNA156c, miRNA156d, miRNA156e, miRNA159, miRNA160, miRNA166a, miRNA166b, miRNA167a, miRNA167b, miRNA168, miRNA169, miRNA172a, miRNA172b, miRNA319a, miRNA319b, miRNA319c, miRNA396a, miRNA396b, miRNA398a, miRNA398b.

miRNA precursors derived from Medicago truncatula contemplated for use in the invention are miRNA156, miRNA160, miRNA162, miRNA166, miRNA169a, miRNA169b, miRNA171, miRNA319, miRNA393, miRNA395a, miRNA395b, miRNA395 c, miRNA395 d, miRNA395 e, miRNA395 f, miRNA395g, miRNA395h, miRNA395i, miRNA395j, miRNA395k, miRNA3951, miRNA395m, miRNA395n, miRNA395o, miRNA395p, miRNA399a, miRNA399b, miRNA399c, miRNA399d, miRNA399e.

miRNA precursors derived from Physcomitrella patens contemplated for use in the invention are miRNA156a, miRNA319a, miRNA319b, miRNA319c, miRNA319d, miRNA319a, miRNA390a, miRNA390b, miRNA390c, miRNA533a, miRNA533b, miRNA534, miRNA535a, miRNA535b, miRNA535c, miRNA535d, miRNA536, miRNA537a, miRNA537b, miRNA538a, miRNA538b, miRNA538c, miRNA1210, miRNA1211, miRNA1212, miRNA1213, miRNA1214, miRNA1215, miRNA1216, miRNA1217, miRNA1218, miRNA1219a, miRNA1219b, miRNA1219c, miRNA1219d, miRNA1220a, miRNA1220b, miRNA1221, miRNA1222, miRNA1223.

miRNA precursors derived from Populus trichocarpa contemplated for use in the invention are miRNA156a, miRNA156b, miRNA156c, miRNA156d, miRNA156e, miRNA156f, miRNA156g, miRNA156h, miRNA156i, miRNA156j, miRNA156k, miRNA159a, miRNA159b, miRNA159c, miRNA159d, miRNA159e, miRNA159f, miRNA160a, miRNA160b, miRNA160c, miRNA160d, miRNA160e, miRNA160f, miRNA160g, miRNA160h, miRNA162a, miRNA162b, miRNA162c, miRNA164a, miRNA164b, miRNA164c, miRNA164d, miRNA164e, miRNA164f, miRNA166a, miRNA166b, miRNA166c, miRNA166d, miRNA166e, miRNA166f, miRNA166g, miRNA166h, miRNA166i, miRNA166j, miRNA166k, miRNA1661, miRNA166m, miRNA166n, miRNA166o, miRNA166p, miRNA166q, miRNA167a, miRNA167b, miRNA167c, miRNA167d, miRNA167e, miRNA167f, miRNA167g, miRNA167h, miRNA168a, miRNA168b, miRNA169a, miRNA169aa, miRNA169ab, miRNA169ac, miRNA169ad, miRNA169ae, miRNA169af, miRNA169b, miRNA169c, miRNA169d, miRNA169e, miRNA169f, miRNA169g, miRNA169h, miRNA169i, miRNA169j, miRNA169k, miRNA1691, miRNA169m, miRNA169n, miRNA169o, miRNA169p, miRNA169q, miRNA169r, miRNA169s, miRNA169t, miRNA169u, miRNA169v, miRNA169w, miRNA169x, miRNA169y, miRNA169z, miRNA171a, miRNA171b, miRNA171c, miRNA171d, miRNA171e, miRNA171f, miRNA171g, miRNA171h, miRNA171i, miRNA171j, miRNA171k, miRNA172a, miRNA172b, miRNA172c, miRNA172d, miRNA172e, miRNA172f, miRNA172g, miRNA172h, miRNA172i, miRNA319a, miRNA319b, miRNA319c, miRNA319d, miRNA319e, miRNA319f, miRNA319g, miRNA319h, miRNA319i, miRNA390a, miRNA390b, miRNA390c, miRNA390d, miRNA393a, miRNA393b, miRNA393c, miRNA393d, miRNA394a, miRNA394b, miRNA395a, miRNA395b, miRNA395c, miRNA395d, miRNA395e, miRNA395f, miRNA395g, miRNA395h, miRNA395i, miRNA395j, miRNA396a, miRNA396b, miRNA396c, miRNA396d, miRNA396e, miRNA396f, miRNA396g, miRNA397a, miRNA397b, miRNA397c, miRNA398a, miRNA398b, miRNA398c, miRNA399a, miRNA399b, miRNA399c, miRNA399d, miRNA399e, miRNA399f, miRNA399g, miRNA399h, miRNA399i, miRNA399j, miRNA399k, miRNA3991, miRNA403a, miRNA403b, miRNA403c, miRNA408, miRNA472a, miRNA472b, miRNA473a, miRNA473b, miRNA474a, miRNA474b, miRNA474c, miRNA475a, miRNA475b, miRNA475c, miRNA475d, miRNA476a, miRNA476b, miRNA476c, miRNA477a, miRNA477b, miRNA478a, miRNA478b, miRNA478c, miRNA478d, miRNA478e, miRNA478f, miRNA478h, miRNA478i, miRNA478j, miRNA478k, miRNA4781, miRNA478m, miRNA478n, miRNA478o, miRNA478p, miRNA478q, miRNA478r, miRNA478s, miRNA478u, miRNA479, miRNA480a, miRNA480b, miRNA481a, miRNA481b, miRNA481c, miRNA481d, miRNA481e, miRNA482.

miRNA precursors derived from Saccharum officinarum contemplated for use in the invention are miRNA156, miRNA159a, miRNA159b, miRNA159c, miRNA159d, miRNA159e, miRNA167a, miRNA167b, miRNA168a, miRNA168b, miRNA396, miRNA408a, miRNA408b, miRNA408c, miRNA408d, miRNA408e.

miRNA precursors derived from Sorghum bicolor contemplated for use in the invention are miRNA156a, miRNA156b, miRNA156c, miRNA156d, miRNA156e, miRNA159, miRNA159b, miRNA160a, miRNA160b, miRNA160c, miRNA160d, miRNA160e, miRNA164, miRNA164b, miRNA164c, miRNA166a, miRNA166b, miRNA166c, miRNA166d, miRNA166e, miRNA166f, miRNA166g, miRNA167a, miRNA167b, miRNA167c, miRNA167d, miRNA167e, miRNA167f, miRNA167g, miRNA168, miRNA169a, miRNA169b, miRNA169c, miRNA169d, miRNA169e, miRNA169f, miRNA169g, miRNA169h, miRNA169i, miRNA171a, miRNA171b, miRNA171c, miRNA171d, miRNA171e, miRNA171f, miRNA172a, miRNA172b, miRNA172c, miRNA172d, miRNA172e, miRNA319, miRNA393, miRNA394a, miRNA394b, miRNA395 a, miRNA395b, miRNA395 c, miRNA395 d, miRNA395 e, miRNA395f, miRNA396a, miRNA396b, miRNA396c, miRNA399a, miRNA399b, miRNA399c, miRNA399d, miRNA399e, miRNA399f, miRNA399g, miRNA399h, miRNA399i.

Endogenous plant miRNAs exhibit a high level of complementarity between the targeting region of the miRNA molecule and the target mRNA molecule. “Endogenous” as used herein refers to material that is derived from the plant being considered. “Complementarity” as used herein refers to the percentage of complementary base pairings that exist between two nucleic acid molecules. This includes DNA to DNA, DNA to RNA, and RNA to RNA base pairings.

Artificial miRNAs (amiRNAs) can be designed to target specific mRNA transcripts. To achieve this, the nucleotide sequence of the precursor miRNA, encoding the targeting region of the mature miRNA, is manipulated so that it is complementary, or partially complementary, to the mRNA sequence of the transcript of the gene to be down-regulated. This allows for the amiRNA to associate with the target mRNA and prevent its translation. Once the mature amiRNA associates with its target mRNA molecule, the target mRNA is usually degraded by endogenous enzymes, or translation is prevented. Artificial miRNA precursor molecules can be designed to target specific mRNA molecules using the internet-based artificial miRNA designer program WMD2 available online from Weigel World.

The WMD2 program incorporates several parameters important for target selection by natural plant miRNAs, namely the perfect pairing of the 5′ portion of the miRNA (position 2 to 12), no mismatches at the presumptive cleavage site (position 10 and 11), no clusters of two mismatches to the 3′ portion of the miRNA, introduction of one to three mismatches into the 3′ portion, uridine at position 1 and if possible adenine at position 10. Furthermore, BLAST searches of the genomic sequences of the relevant plant species, using the resultant amiRNA sequences, excludes the amiRNAs that may target the off-target transcripts.

“Target” as used herein refers to either the gene that is selected, or “targeted”, for down-regulation, or translational repression, or its corresponding mRNA transcript molecule, which in the case of the present invention is a gene involved in pollen development. The mature artificial miRNA molecule will bind to the target mRNA (or mRNA of the target gene), thus preventing translation of the mRNA into a polypeptide product.

“Targeting region” as used herein refers to the nucleotides of the mature amiRNA molecule that are complementary (defined by the WMD2 program) to the target mRNA.

The invention relates to a reversible transgenic plant male sterility system. “Plant male sterility” as used herein refers to a condition in which a plant lacks the ability to produce pollen, lacks the ability to release pollen, or produces pollen that is incapable of fertilising the female reproductive cells of a flower. The level of male sterility induced by the use of the invention is 100%.

“Male fertility” as used herein refers to the ability to produce and/or release pollen capable of fertilising the female reproductive cells of a flower.

“Native” as used herein means material that is derived from the plant being considered and not altered from its naturally occurring form, whereas “heterologous” means material that is derived from a different source or that has been altered from its naturally occurring form. Such alterations may include deletions, substitutions, or additions as long as they do not change the function of the molecule being altered.

The targeting region of the mature amiRNA molecule may be derived from a sequence that is native to the plant being considered, or an equivalent sequence from a heterologous source, or even from a synthetically generated sequence. In the present invention the targeting region is derived from a gene involved in pollen development. While it is preferable that the sequence of the targeting region is partially complementary (defined by the WMD2 program) to the target mRNA, artificial miRNAs may possess targeting regions that are fully complementary to the target sequence, or targeting regions that are fully complementary to the target sequence only in the first ten to fifteen bases from the 5′ end of the amiRNA, while still retaining the ability to bind to their target mRNAs. The partially complementary amiRNAs generated using the WMD2 program generally contain a mismatch at the first base in 5′ and one to two mismatches at the 3′. Additional mismatches at the 3′ may be included.

“Gene involved in pollen development” as used herein refers to any gene that is involved in the pollen development process at any stage. This includes transcription factors that control the expression of other genes involved in the pollen development process, genes involved in anther development, genes encoding proteins that are directly involved with the formation of pollen, and genes involved in pollen shedding. Some genes contemplated by the invention include transcription factors, such as those of the R2-R3 family of transcription factors or those of the MYB class of transcription factors, particularly those expressed solely in the tapetum. Examples of MYB genes with a specific role in anther or pollen development that may be suitable for use in the invention are AID1 from rice, ZmMYBP2 from maize, NtMYBAS1 and NtMYBAS2 from tobacco, and AtMYB33, AtMYB65, AtMYB26, AtMYB103 and AtMYB32 from Arabidopsis. Due to the homology between the genes in different species the inventors propose that MYB genes from one species may be used in constructs used to transform other species. For example, the AtMYB103 amiRNA construct may be used in both Arabidopsis and canola (Brassica napus). AtMYB103 from Arabidopsis is of particular interest due to its expression being limited to the tapetum.

The invention provides a method of producing a male sterile transgenic plant comprising transforming a plant with an isolated nucleic acid encoding a precursor amiRNA targeted to a gene involved in pollen development.

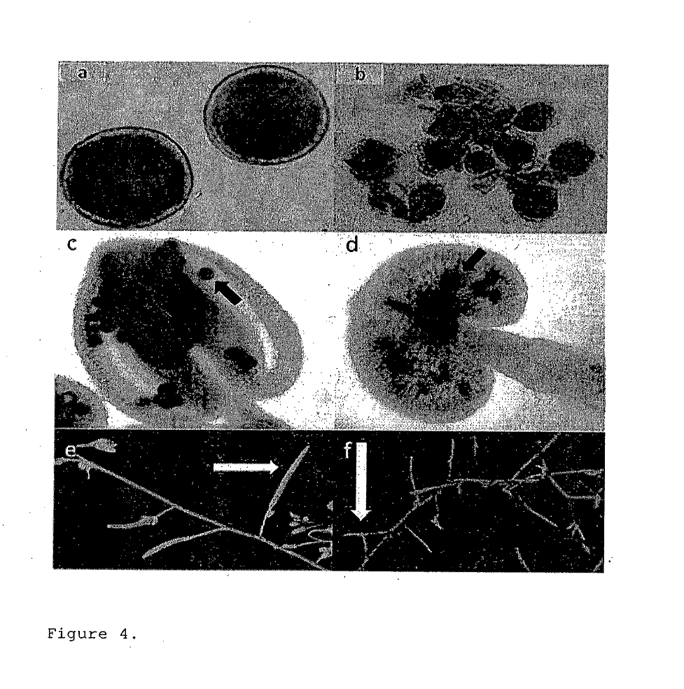

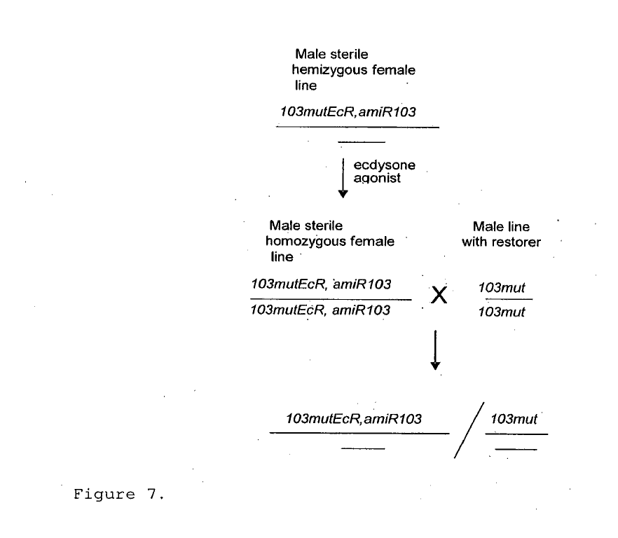

In an embodiment, the male sterile transgenic plant comprising an isolated nucleic acid encoding a precursor amiRNA targeted to a gene involved in pollen development, or a gene coding for a transcription factor involved in pollen development, further comprises a second nucleic acid encoding a mutated copy of the same gene involved in pollen development fused in frame to a ligand binding domain of the ecdysone receptor (inducible activator) (FIG. 7). For a gene other than a transcription factor gene, an inducible promoter (e.g. an ecdysone inducible promoter) is used to drive a mutated copy of the same gene involved in pollen development. This allows for the production of a homozygous male sterile plant line that can be maintained through ecdysone agonist application. The mutated copy of the gene involved in pollen development is not targeted by the amiRNA because it contains point mutations in the target site. The EcR has advantages over other receptors as it is the target of several commercially available insecticides (ecdysone agonists). These non-steroidal ecdysone agonists have been used in many field applications. The protein produced from the mutated gene involved in pollen development fused to the ecdysone receptor remains inactive in the absence of ecdysone agonist, but is activated to restore male fertility following agonist application and thus transform hemizygous male sterile plants into homozygous plants. Homozygous male sterile plants could not be obtained without the ecdysone inducible activator. Hence, a male sterile and homozygous female line could be generated and maintained with agonist application (FIG. 4). In a hybrid seed production field, all female plants are male sterile and no application would be required. It is anticipated that an inducible promoter could be used instead of the ecdysone receptor to achieve the same outcome.

In an embodiment, the amiR103 precursor, MYB103mut (restorer) and inducible activator MYB103mutEcR are driven by MYB103 promoter (FIG. 7). MYB103mut (restorer) contains point mutations in the nucleotide sequence targeted by amiRNA103. The inducible activator (MYB103muyEcR) consists of the restorer fused in frame to the EcR. The inducible activator and amiR103 precursor are introduced into a male sterile line together or separately. The male sterility caused by amiR103 is reversed by the agonist-activated MYB103mutEcR. The resultant male sterile and homozygous line is then pollinated by a male fertile line containing a restorer MYB103mut. For a gene other than a transcription factor gene, an inducible promoter (eg, an ecdysone inducible promoter) is used to drive a mutated copy of the same gene involved in pollen development.

In another embodiment, inducible male sterility can be achieved using an inducible promoter (native, synthetic or chimeric) driving an isolated nucleic acid encoding a precursor amiRNA targeted to a gene involved in pollen development. The inducible promoter is activated by an inducer binding to its receptor (native, synthetic or chimeric). The receptor-inducer complex binds to and activates the inducible promoter. The receptor gene is driven by an anther specific promoter.

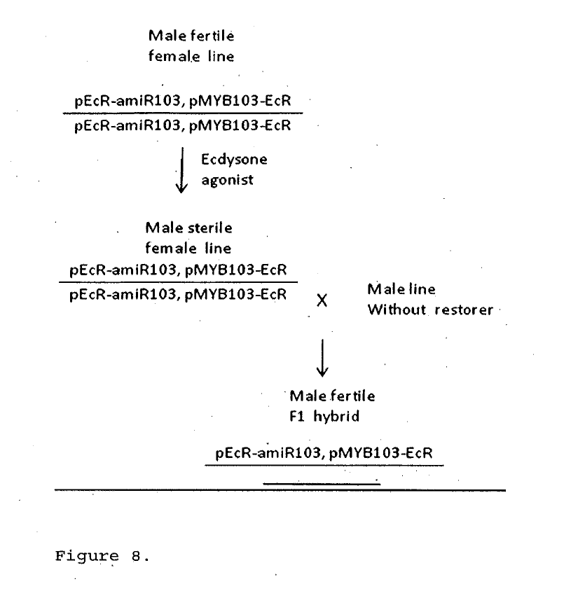

The artificial miRNA (amiR103) precursor targeting MYB103 is driven by an ecdysone inducible promoter pEcR (native or synthetic or chimeric promoter) (pEcR-amiR103). The ecdysone receptor ligand binding domain is fused in frame with a DNA binding domain capable of binding to the inducible promoter and is driven by MYB103 promoter (pMYB103-EcR). The homozygous male fertile plants become male sterile when treated with an ecdysone agonist and pollinated with pollen from donor plants without a restorer. The resultant F1 hybrid plants are male fertile (FIG. 8).

“Homozygous” as used herein refers to a plant that has two identical alleles for the gene being considered. Genomes possess two copies of each gene, one on each member of a chromosome pair, with one chromosome coming from the female parent's genome and one copy coming from the male parent's genome. These two copies are known as alleles, and due to their different origins, they can possess slightly different sequences.

The invention also provides a method of producing a male fertility restorer transgenic plant that is capable of reversing male sterility in the hybrid progeny of a plant in which male sterility has been induced by amiRNA down-regulation of a gene involved in pollen development comprising transforming a plant with an isolated nucleic acid encoding a mutated copy of the same gene involved in pollen development that was targeted by the amiRNA in the male sterile transgenic plant. The mutated copy of the gene involved in pollen development comprises at least one mutation, said mutation preferably being a conservative mutation that alters the nucleotide sequence without altering the amino acid sequence. This mutation has the consequence that the amiRNA is no longer complementary enough to the target mRNA, meaning that the amiRNA cannot associate with the target mRNA and down-regulate the gene.

The invention also provides a further method of producing a male fertility restorer transgenic plant that is capable of reversing male sterility in the hybrid progeny of a plant in which male sterility has been induced by amiRNA down-regulation of a gene involved in pollen development, comprising transforming a plant with multiple copies of an isolated nucleic acid encoding the same gene involved in pollen development that was targeted by the amiRNA in the male sterile transgenic plant, or a copy of an isolated nucleic acid encoding the same gene involved in pollen development that was targeted by the amiRNA in the male sterile transgenic plant under the control of a strong anther specific promoter. The expression of the multiple copies of the gene involved in pollen development, or the copy of the gene involved in pollen development under the control of a strong promoter has the consequence that the amiRNA down-regulation is overwhelmed and is no longer capable of inducing 100% male sterility.

The invention provides a male sterile transgenic plant produced by transforming a plant with a nucleic acid encoding an amiRNA targeted to a gene involved in pollen development and/or a male fertility restorer transgenic plant produced by transforming a plant with an isolated nucleic acid encoding a mutated copy of the same gene involved in pollen development that was targeted by the amiRNA in the male sterile transgenic plant, as well as transgenic seed, propagating material or progeny of such transgenic plants.

Also provided is a male fertility restorer transgenic plant, comprising multiple copies of the gene involved in pollen development, or a copy of the gene involved in pollen development under the control of a strong promoter, as well as transgenic seed, propagating material or progeny of such transgenic plants.

Male fertile hybrid progeny seed can be produced by fertilising the male sterile transgenic plant produced by the invention with pollen from a male fertility restorer transgenic plant produced by the invention, and collecting the resulting male fertile hybrid seed.

The invention further provides an isolated nucleic acid encoding a precursor amiRNA encoding an amiRNA targeted to a gene involved in pollen development. This nucleic acid may be operably linked to one or more promoters capable of driving expression of the precursor amiRNA. Both native and heterologous promoters are suitable for use in driving expression of the amiRNA molecule.

By “promoter” is meant a minimal sequence sufficient to direct transcription. “Promoter” is also meant to encompass those promoter elements sufficient for promoter-dependent gene expression controllable for cell-type specific, tissue-specific or inducible by external signals or agents; such elements may be located in the 5′ or 3′ regions of the native gene. A promoter may be constitutive but most preferably is inducible. The promoter may be native to the plant being considered, or it may be heterologous to the plant being considered. For example, the strong BnMYB103 promoter from Brassica napus, or functional equivalents thereof, may be used to drive expression of the precursor amiRNA. The construct may contain one or more than one promoter.

“Nucleic acid” as used herein refers to an oligonucleotide, polynucleotide, nucleotide and fragments or portions thereof, as well as to peptide nucleic acids (PNA), fragments, portions or antisense molecules thereof, and to DNA or RNA of genomic or synthetic origin which can be single-stranded, or double-stranded, and represent the sense or antisense strand. Where “nucleic acid” is used to refer to a specific nucleic acid sequence “nucleic acid” is meant to encompass polynucleotides that encode a polypeptide that is functionally equivalent to that encoded by the recited nucleic acid, e.g., polynucleotides that are degenerate variants, or polynucleotides that encode biologically active variants or fragments of the polypeptide, including polynucleotides having substantial sequence similarity or sequence identity relative to the sequences provided herein.

Similarly, “polypeptide” as used herein refers to an oligopeptide, peptide, or protein. Where “polypeptide” is recited herein to refer to an amino acid sequence of a naturally-occurring protein molecule, “polypeptide” and like terms are not meant to limit the amino acid sequence to the complete, native amino acid sequence associated with the recited protein molecule, but instead is meant to also encompass biologically active variants or fragments, including polypeptides having substantial sequence similarity or sequence identity relative to the amino acid sequences provided herein.

By “isolated” we mean a molecule or compound that is free from material present in nature in the plant from which the nucleic acid molecule is derived, or that is in an environment different from that in which the molecule naturally occurs. “Isolated” is meant to include compounds that are within samples that are substantially enriched for the compound of interest and/or in which the compound of interest is partially or substantially purified.

The invention provides an isolated nucleic acid comprising a mutated copy of a gene involved in pollen development comprising one or more mutations conferring resistance to an amiRNA that would effectively target an un-mutated copy of the same gene.

The “mutations” conferring resistance to the amiRNA may be substitutions, additions, or deletions that result in the amiRNA being unable to associate with the target mRNA, thus preventing amiRNA down-regulation of the target gene. For example, the mutation may be a conservative and, or point mutation in the gene involved in pollen development. The inability of the amiRNA to associate with and down-regulate the target mRNA results in viable pollen production and seed setting in the hybrid progeny.

The invention also provides a vector, host cell and transgenic plant comprising the nucleic acids of the invention. Vectors suitable for the transformation of a wide variety of plants are known and an appropriate vector could be selected by one of skill in the art. The invention encompasses transgenic seed, propagating material or progeny derived from the transgenic plants of the invention.

“Transgenic plant” as used herein refers to a plant that has been manipulated using genetic engineering techniques to alter its genetic content. This alteration may involve the introduction of genetic material that is not native to the host, or to the alteration of the native genetic material of the host.

“Heterosis” as used herein refers to the superior performance of heterozygous hybrid plants over their inbred parents. In order to assess superior performance, average trait values of hybrids, including yield, plant size, speed of development, fertility, resistance to disease and pests along with a long list of biotic and abiotic stresses, are compared to those of the parent plants. Heterosis is also referred to as hybrid vigour.

“Hybrid” as used herein refers to the progeny produced by crossing genetically different parental lines. The progeny of a hybrid cross may be self-pollinated to create inbred plant lines that possess particular desired characteristics. Two different inbred lines may be crossed to produce hybrid seeds.

“Crossing” as used herein refers to the process of pollinating the female part of the flower of a plant with pollen from a different plant. Variations of the term crossing encompass cross-pollinating, cross-breeding, crossed, and cross.

“Parental lines” as used herein refers to the two genetically different plants which are crossed to generate hybrid plants. The parental lines may be inbred and/or male sterile. The parental lines may comprise a restorer of male fertility.

“Inbred” as used herein refers to a plant that has been pollinated with pollen from itself. Variations of the term inbred include selfed, self-pollinated, doubled haploid.

“Progeny” as used herein refers to plants grown from the seeds of a plant. Such plants may be inbred progeny or they may be hybrid progeny.

“F1” as used herein refers to the first generation of progeny produced from the crossing of two genetically different parental lines. The progeny of these genetically different parental lines will be a new, uniform variety with specific characteristics from either or both parents. To produce consistent F1 hybrids, the original cross must be repeated each season.

“Wild type” as used herein refers to a plant that has not had its genome manipulated by either the use of breeding techniques such as crossing and selfing, or by genetic engineering.

For the purposes of this specification it will be clearly understood that the word “comprising” means “including but not limited to” and that the word “comprises” has a corresponding meaning.

It will be clearly understood that, although a number of prior art publications may be referred to herein, this reference does not constitute an admission that any of these documents forms part of the common general knowledge in the art, in Australia or in any other country.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

Embodiments of the present invention will now be described in the following non-limited examples.

### EXAMPLES

### Example 1

### An artificial miRNA (amiRNA 1) Targeting Arabidopsis AtMYB103 and Canola BNMYB103 Transcripts

The endogenous miR319a Arabidopsis precursor was used as a backbone. This precursor does not target AtMYB103 naturally so the endogenous 21 base miRNA sequence had to be changed into a new 21 base amiRNA sequence targeting Arabidopsis AtMYB103 and canola BnMYB103 transcripts.

The WMD2 tool does not include a Brassica napus database, therefore the amiRNA sequence was designed manually by selecting 21 base target sequences that are identical between Arabidopsis AtMYB103 and canola BnMYB103 genes. The target sequences are downstream from the sequences coding for the MYB domains (GACGAGATGCTTCACTTGCTC, position 982 bp downstream from the translational start of AtMYB103).

The amiRNA (TAGCAAGTGAAGCATCTCGGC) was designed following the developer's rules. The first base at the 5′ end of the reverse complementary sequence of the selected 21 base Brassica napus endogenous sequence, a Guanine, was substituted with a Thymine and the second last base at the 3′ end of the reverse complementary sequence of the selected 21 base Brassica napus endogenous sequence, a Thymine, was substituted with a Guanine.

Based on the amiRNA sequence, the WMD2 designer predicts four oligonucleotide sequences which are used to engineer the amiRNA into the miRNA and miRNA-complementary (GAGCAAGTGAAGCATCTCGTC, amiRNA*) regions of the endogenous miR319a precursor by overlap PCR. The Brassica napus BnMYB103 promoter was chosen to drive the precursor expression because it is stronger than the AtMYB103 promoter.

### Example 2

### Preparation of a First amiR103/319a Construct (amiRNA 1)

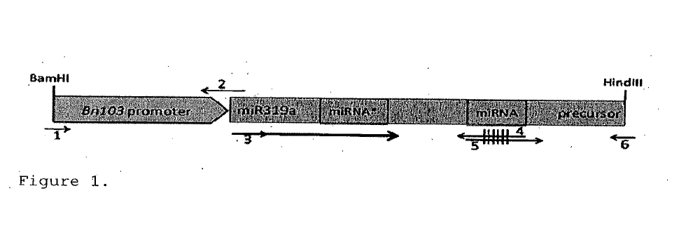

The miR103/319a precursor construct (amiRNA 1) was designed to target the MYB103 transcripts of Arabidopsis and canola. The amiRNA103 precursor was driven by the canola BnMYB103 promoter. The amiRNA 1 was constructed using overlap PCR. Two overlap regions are present; first is between promoter and precursor (primers were designed to allow the overlap); the second is the amiRNA region (FIG. 1).

Six primers were designed and three separate PCR reactions were carried out using high-fidelity DNA polymerase (Ex Taq DNA polymerase) to minimize any mutations in the precursor, especially in the miRNA/miRNA-complementary region. In the first reaction a full Bn103 promoter, not including the gene starting codon ATG, and 25 bp of the precursor were amplified, using primers 1 and 2 (BnF and miRNAR), and Bn103c DNA as a template. In the second reaction primers 3 and 4 (BnMuF and BnMuR), were used to amplify and overlap the end of the promoter and a large part of the precursor region containing amiRNA-complementary. In the third reaction, primers 5 and 6 (BnMuF1 and miRNAPR), amplified the amiRNA region, including the end of the precursor. PCR-Script plasmid containing the precursor was used as a template for both reactions. Purified PCR products from the three reactions served as a template for the 4^{th} PCR reaction in which primers 1 and 6 containing BamHI and HindIII cut sites, respectively, were used to amplify the entire construct.

The amiR103/319a precursor overlap PCR product was ligated into pPCR-Script plasmid, containing the amiR319a backbone and transformed into E. coli cells. Positive transformants were sequenced to verify that no undesired mutations had been introduced. The amiR103/319a precursor was ligated into pCAMBIA 1380 plasmid and the resulting plasmid used to transform Agrobacterium tumefaciens AGL1 or GV3101 strain (FIG. 1).

### Example 3

### Preparation of a Second amiR103/319a Construct (amiRNA 2)

A second amiRNA construct (amiRNA 2) was developed to target a second sequence in the AtMYB103 and BnMYB103 genes (CTCGCATCTAATGGCAGAGAT, position 876 bp downstream from the translational start site of AtMYB103). The target sequences are downstream from the sequences coding for the MYB domains and are identical between AtMYB103 and BnMYB103 genes.

The amiRNA 2 (TTCTCTGCCATTAGATGGCAG) and amiRNA* (CTACCATCTAATGCCAGAGAT) were designed following the relevant rules in WMD2.

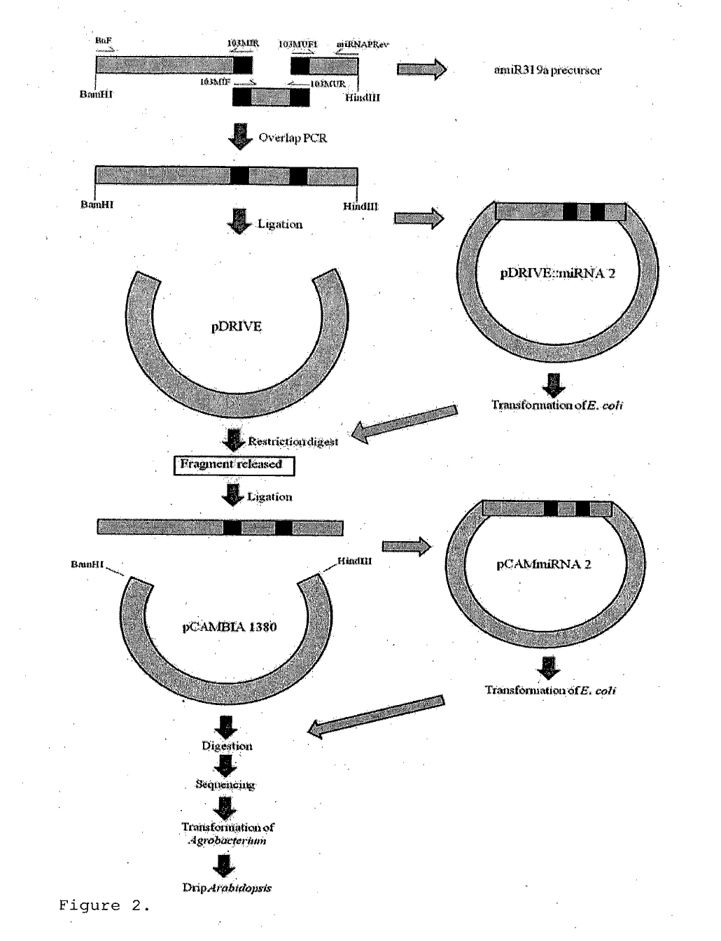

The amiRNA 2 construct is comprised of three fragments (FIG. 2). Each of these fragments was amplified (FIG. 2) from the plasmid template pCAMmiRNA (amiRNA 1 construct) using primers that change the amiRNA 1 sequence to the amiRNA 2 sequence. The three fragments were amplified using primers 1 and 11 (BnF and 103MIR), primers 12 and 13 (103MIF and 103MUR_, and primers 14 and 15 (103MUF1 and MIRNAPRev), respectively.

High-fidelity DNA polymerase (TaKaRa™ ExTaq) was employed to produce an overlap fragment from the three fragments. The fragment, containing an additional adenine residue at the 3′ end, was ligated into pDRIVE which contains a 3′-Uracil overhang. The amiRNA 2 insert was released from pDRIVE by restriction digest and ligated into the BamH1-HindIII sites of binary vector pCAMBIA 1380 and the resulting plasmid was named pCAMmiRNA2. E. coli cells were transformed with pCAMmiRNA2. Subsequently, plasmid DNA from three different clones was digested to confirm the presence of the amiRNA 2 construct and the inserts were sequenced. One of the confirmed clones was used to transform Agrobacterium tumefaciens GV3101 strain for plant transformation.

### Example 4

### Preparation of a Third amiR103/319a Construct Containing Two amiR103/319a Precursors (amiRNA 1-2)

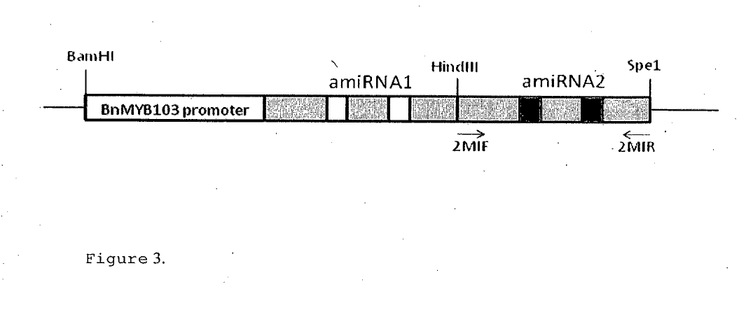

A third amiRNA construct (amiRNA 1-2) containing the amiRNA 1 and amiRNA 2 precursors in tandem and driven by the canola BnMYB103 promoter was developed (FIG. 3). The two mature amiRNAs generated from the construct target the amiRNA 1 sequence and the amiRNA 2 sequence of the AtMYB103 and BnMYB103 transcripts. Accordingly, the third construct should be more efficient in inducing male sterility than either the amiRNA 1 or amiRNA 2 constructs were individually. The amiRNA 2 precursor fragment was amplified from the amiRNA 2 construct using primer 20 2MIF (containing a HindIII site) and primer 21 2MIR (containing a Spe1 site). The fragment was digested with HindIII and Spe1 and ligated into the HindIII and Spe1 sites of the amiRNA 2 construt. The insert was sequenced.

### Example 5

### Transformation of A. thaliana by Agrobacterium-Mediated Transformation

Wild type Arabidopsis flower buds were dripped several times using Agrobacterium tumefaciens GV3101 containing the amiRNA 1 construct or the amiRNA 2 construct. Seeds were harvested and spread onto selection plates containing timetin and hygromycin. Hygromycin was used to select for positive plant transformants.

A total of 78 lines were obtained for the amiRNA 1 construct. Ten lines were completely male sterile and five lines were partially male sterile. Extraction and PCR analysis of genomic DNA from the young leaves of the complete and partial male sterile lines were performed to confirm the presence of the transgenes. The lines examined contained the amiRNA 1 construct.

### Example 6

### Evaluation of the Male Fertility Status of Transgenic Plants for amiRNA 1

Five lines (amiRNA 1) were partially male sterile and ten lines displayed complete male sterility (ie, all the plants in each complete male sterile lines are 100% male sterile). The wild type expressed no abnormalities while the male sterile line had aborted siliques and set no seeds (FIG. 4). No other phenotypic differences could be observed between the two lines other then the differences in pollen morphology and seed setting. The male sterile plants displayed complete female fertility as normal seed set occurred using wild-type donor pollen.

The anthers of wild type closed flowers released mature, regularly shaped, fertile pollen with intact cytoplasm. The male sterile, amiRNA 1 lines lacked pollen grains in the closed flower buds. In partially open flowers, anthers produced pollen, which formed aggregates which could not be released from the anther. Examination of fully open flower anthers revealed the aborted pollen grains had been released. Closer examination of individual pollen grains revealed abnormal pollen shapes and little cytoplasmic content (FIG. 4).

### Example 7

### Preparation of a Fertility Restorer 1 Construct (Restorer 1)

Male sterility is often difficult to maintain in hybrid seed production. The system is complex and requires the presence of three separate components: the source of male sterility, the availability of maintainer lines and restoration of male fertility in hybrids whose harvested product is seed or fruit. Therefore, a male fertility restorer line is essential in plant breeding programs.

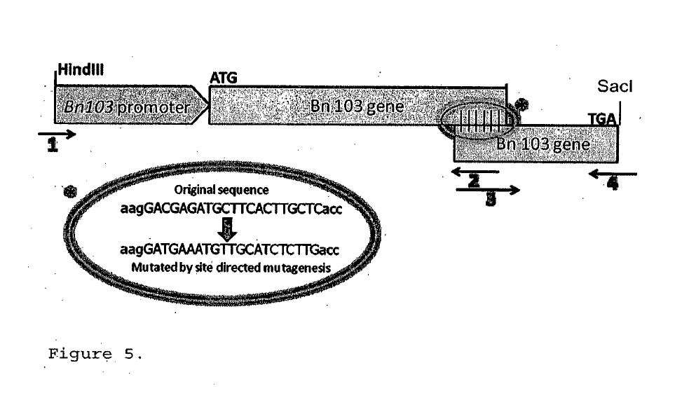

The Restorer 1 construct was created by overlap PCR amplification of canola BnMYB103 (including the promoter and coding region) (FIG. 5). Primers were designed in order to mutate a specific 21 base sequence in the third exon of the Brassica napus BnMYB103 gene. It is the same sequence initially used to create the amiRNA 1 construct used for induction of male sterility. The sequence was mutated by the PCR primers which involved maximum number of nucleotide substitutions while leaving the amino acid sequence unchanged. This ensures that the correct protein is still produced. Accordingly, the Restorer 1 transcript should not be targeted by amiRNA 1. Two independent PCR reactions were carried out using Phusion, high-fidelity DNA polymerase. Primers 7 and 8 (Mut For1 and Mut Rev1), or primers 9 and 10 (Mut For2 and Mut Rev2), were designed to amplify the promoter and the gene, including the mutated sequence (FIG. 5).

The mutated region is where the overlap occurs. PCR was performed to create the two independent fragments of 1724 bp and 492 bp. The two bands were purified and used as templates for the overlap PCR. Primers 7 and 10 were then used to amplify the entire fragment giving the expected size of 2216 bp. The overlap PCR product was ligated into the pDRIVE plasmid and sequenced. The HindIII-SacI fragment was released by restriction digest and ligated into the HindIII-SacI sites of the pBI101 cloning vector. The Restorer 1 construct was transformed into Agrobacterium tumefaciens GV3101.

### Example 8

### Preparation of a Fertility Restorer 2

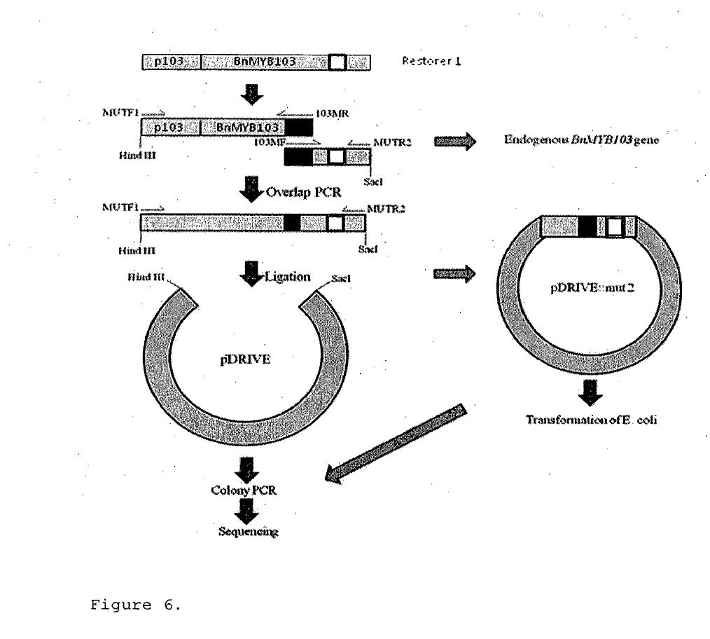

The Restorer 2 construct was created to restore male fertility to plants expressing amiRNA 1 or amiRNA 2 or amiRNA 1-2. The Restorer 1 construct was used as a template for Restorer 2 construction. Two fragments were amplified from the pBI101+mut plasmid (restorer 1) using KAPA HiFi DNA Polymerase with primers 16 and 17 (MUTF1 and 103MR), and primers 18 and 19 (103MF and MUTR2). The overlapping regions, represented by the black box in FIG. 6, contain a mutated gene sequence so that it codes for the same amino acids as the endogenous gene but changes the codons so that the amiRNA 2 can no longer target the transcript. The two fragments were annealed to one another in an overlap PCR using high-fidelity DNA polymerase (TaKaRa™ ExTaq). The area represented by the open box in FIG. 6 contains the mutated sequence of restorer 1 which prevents the amiRNA 1 from binding. Hence, the restorer 1 transcript should not be targeted by amiRNA 1, amiRNA 2 or amiRNA 1-2. The Restorer 2 construct was ligated into pDRIVE and used to transform E. coli. Eight colonies were analysed for the presence of the transgene by colony PCR and all were positive. The inserts in two plasmids were sequenced. The Restorer 2 construct was transformed into Agrobacterium tumefaciens GV3101 for plant transformation.

### Example 9

### Transformation of A. thaliana by Agrobacterium-Mediated Transformation

Agrobacterium (GV3101 strain) was transformed with the Restorer 1 construct and wild type Arabidopsis plants were transformed using several rounds of dripping onto unopened flower buds. Seeds were collected and spread onto GM selection plates, containing timetin and hygromycin and plants transgenic for the Restorer 1 construct were selected.

SEQUENCES
SEQ ID NO: 1
miR319a precursor (Pre-miR)(AT4G23713)
AGAGAGAGCTTCCTTGAGTCCATTCACAGGTCGTGATATGATTCAATTAG
CTTCCGACTCATTCATCCAAATACCGAGTCGCCAAAATTCAAACTAGACT
CGTTAAATGAATGAATGATGCGGTAGACAAATTGGATCATTGATTCTCTT
TGATTGGACTGAAGGGAGCTCCCTCT
SEQ ID NO: 2
miR319b precursor (Pre-miR) (AT5G41663)
AGAGAGCTTTCTTCGGTCCACTCATGGAGTAATATGTGAGATTTAATTGA
CTCTCGACTCATTCATCCAAATACCAAATGAAAGAATTTGTTCTCATATG
GTAAATGAATGAATGATGCGAGAGACAAATTGAGTCTTCACTTCTCTATG
CTTGGACTGAAGGGAGCTCCCT
SEQ ID NO: 3
miR319c precursor (Pre-miR) (AT2G40805)
TAGATATAGAAGGAGATTCTTTCAGTCCAGTCATGGATAGAAAAAGAAGA
GGGTAGAAATATCTGCCGACTCATCCATCCAAACACTCGTGGTAGAGAAA
CGATAAATTTAAACCGCAGTGACTGTGTGAATGATGCGGGAGATATTTTT
GATCCTTCTTTATCTGTGTTTGGACTGAAGGGAGCTCCTTCTTTTTCTA
SEQ ID NO: 4
miR159a precursor (Pre-miR)(AT1G73687)
TAGAGCTCCTTAAAGTTCAAACATGAGTTGAGCAGGGTAAAGAAAAGCTG
CTAAGCTATGGATCCCATAAGCCCTAATCCTTGTAAAGTAAAAAAGGATT
TGGTTATATGGATTGCATATCTCAGGAGCTTTAACTTGCCCTTTAATGGC
TTTTACTCTTCTTTGGATTGAAGGGAGCTCTA
SEQ ID NO: 5
miR159b precursor (Pre-miR) (AT1G18075)
AAGAGCTCCTTGAAGTTCAATGGAGGGTTTAGCAGGGTGAAGTAAAGCTG
CTAAGCTATGGATCCCATAAGCCTTATCAAATTCAATATAATTGATGATA
AGGTTTTTTTTATGGATGCCATATCTCAGGAGCTTTCACTTACCCCTTTA
ATGGCTTCACTCTTCTTTGGATTGAAGGGAGCTCTT
SEQ ID NO: 6
miR159c precursor (Pre-miR)(AT2G46255)
GTGTAACAGAAGGAGCTCCCTTCCTCCAAAACGAAGAGGACAAGATTTGA
GGAACTAAAATGCAGAATCTAAGAGTTCATGTCTTCCTCATAGAGAGTGC
GCGGTGTTAAAAGCTTGAAGAAAGCACACTTTAAGGGGATTGCACGACCT
CTTAGATTCTCCCTCTTTCTCTACATATCATTCTCTTCTCTTCGTTTGGA
TTGAAGGGAGCTCCTTTTCTTCTTC
SEQ ID NO: 7
Primer 1 - BnF
CACAGGATCCGTGGGGGTCCTGTAATTGACTC
SEQ ID NO: 8
Primer 2 - miRNA R
CAGCGAGTATTAAGTGTATGAACATGTGTAATATGCGTCCGAGCGTGTGT
TTGTCTTTCTTTCTAGTTTTTATCTCTCGATCG
SEQ ID NO: 9
Primer 3 - BnMu F
GTTCATACACTTAATACTCGCTGTTTTGAATTGATGTTTTAGGAATATAT
ATGTAGAGCAGAGATGCTTCTCTTGCTTTCACAGGTCGTGATATGATTC
SEQ ID NO: 10
Primer 4 - BnMu R
GCCGAGATGCTTCACTTGCTATCAAAGAGAATCAATGATCCAA
SEQ ID NO: 11
Primer 5 - BnMuF1
TAGCAAGTGAAGCATCTCGGCTCTCTCTTTTGTATTCCAATTTTCTTGA
SEQ ID NO: 12
Primer 6 - miRNA P R
TGATAAGCTTCATGGCGATGCCTTAAATAAAGATA
SEQ ID NO: 13
Primer 7 - Mut For1
CACAAAGCTTGTGGGGGTCCTGTAATTGACTC
SEQ ID NO: 14
Primer 8 - Mut Rev1
CAAGAGATGCAACATTTCATCCTTGAAACATCCGAGGGCAGCTT
SEQ ID NO: 15
Primer 9 - Mut For2
GATGAAATGTTGCATCTCTTGACCAAGAAACGTGTTGATCTAAAC
SEQ ID NO: 16
Primer 10 - Mut Rev2
CTGCGAGCTCTCAAACCACATGATTAATGAGATCA
SEQ ID NO: 17
Primer 11 - 103MIR
ATCTCTGGCATTAGATGGTAGTCTACATATATATTCCTAAAACATC
SEQ ID NO: 18
Primer 12 - 103MIF
CTACCATCTAATGCCAGAGATTCACAGGTCGTGATATG
SEQ ID NO: 19
Primer 13 - 103MUR
CTGCCATCTAATGGCAGAGAATCAAAGAGAATCAATGATCCAA
SEQ ID NO: 20
Primer 14 - 103MUF1
TTCTCTGCCATTAGATGGCAGTCTTCTTTTTGTATTCCAA
SEQ ID NO: 21
Primer 15 - miRNAPREV
TGATAAGCTTCATGGCGATGCCTTAAATAAAGATA
SEQ ID NO: 22
Primer 16 - MUTF1
CACAAAGCTTGTGGGGGTCCTGTCATTGACTC
SEQ ID NO: 23
Primer 17 - 103MR
ATTTCAGCCATCAAGTGTGAAAAAGGCTTGTGGGTTACGGGA
SEQ ID NO: 24
Primer 18 - 103MF
TTTTCACACTTGATGGCTGAAATAACCACTACACTCAATCCT
SEQ ID NO: 25
Primer 19 - MUTR2
CTG CGAGCTCTCAAACCACATGATTAATGAGATCA
SEQ ID NO: 26
Primer 20 - 2MIF
AACTAGTCATGGCGATGCCTTAAATAAAG
SEQ ID NO: 27
Primer 21 - 2MIR
TAAGCTTCAAACACACGCTCGGACGCAT

1.-55. (canceled)

56. A reversible transgenic plant male sterility system wherein the male sterility is induced by amiRNA.

57. A method for producing male sterility in a plant comprising expressing in said plant an amiRNA from a male sterility construct comprising an isolated nucleic acid encoding a precursor amiRNA encoding an amiRNA targeted to a gene involved in pollen development.

58. A reversible transgenic plant male sterility system comprising:
(i) a male sterility construct comprising an isolated nucleic acid encoding a precursor amiRNA encoding an amiRNA targeted to a gene involved in pollen development; and
(ii) a male fertility restorer construct comprising an isolated nucleic acid encoding a mutated copy of said gene involved in pollen development, said mutated copy comprising at least one mutation conferring resistance to said amiRNA targeted to said gene involved in pollen development;
optionally the system further comprising:
(iii) an inducible male fertility activator construct (inducible restorer) comprising an isolated nucleic acid encoding a mutated copy of said gene involved in pollen development, said mutated copy comprising at least one mutation conferring resistance to an amiRNA targeted to said gene involved in pollen development, wherein said mutated copy comprising at least one mutation conferring resistance to an amiRNA is fused in frame to a ligand binding domain of an ecdysone receptor.

59. The reversible transgenic plant male sterility system of claim 58, wherein the male sterility construct further comprises:
(i) an inducible promoter operably linked to the isolated nucleic acid encoding a precursor amiRNA targeted to a gene involved in pollen development; optionally wherein the inducible promoter is
(a) native, or
(b) synthetic, or
(c) chimeric, or
(d) is an ecdysone inducible promoter.

60. The reversible transgenic plant male sterility system of claim 58, wherein the plant male sterility is reversed in hybrid progeny seed by fertilising a male sterile transgenic plant generated using the male sterility construct with pollen from a male fertility restorer transgenic plant generated using the male fertility restorer construct.

61. The reversible transgenic plant male sterility system of claim 58, wherein the plant male sterility is reversed in hybrid progeny seed by fertilising a male sterile transgenic plant, generated using the male sterility construct comprising the inducible promoter, with pollen from a wild-type plant.

62. A method of producing a male sterile transgenic plant comprising transforming the plant with an isolated nucleic acid encoding a precursor amiRNA targeted to a gene involved in pollen development; optionally
wherein the plant is transformed with a second isolated nucleic acid encoding a gene involved in pollen development said gene comprising at least one mutation conferring resistance to an amiRNA targeted to said gene involved in pollen development,
wherein said second nucleic acid is fused in frame to a ligand binding domain of the ecdysone receptor.

63. The method of claim 59, wherein the precursor amiRNA further comprises a constitutive promoter operably linked to the precursor amiRNA, optionally
wherein the constitutive promoter is a native promoter or a heterologous promoter.

64. The method of claim 62, wherein the precursor amiRNA further comprises an inducible promoter operably linked to the precursor amiRNA; optionally
wherein the inducible promoter is
(a) native, or
(b) synthetic, or
(c) chimeric, or
(d) is an ecdysone inducible promoter.

65. The method of claim 62, wherein the precursor amiRNA contains a target sequence that is
(i) native to the plant being considered, or
(ii) heterologous to the plant being considered, or
(iii) synthetically generated, or
(iv) partially complementary to the target sequence, or
(v) fully complementary to the target sequence, or
(vi) fully complementary to the target sequence in the first ten to fifteen bases from the 5′ end of the amiRNA.

66. A method of producing a male fertility restorer transgenic plant capable of restoring fertility to the hybrid progeny of a male sterile plant produced by the method of claim 62 comprising transforming a plant with an isolated nucleic acid encoding a mutated copy of a gene involved in pollen development said mutated copy comprising at least one mutation conferring resistance to an amiRNA targeted to said gene involved in pollen development.

67. The method of claim 62, wherein the at least one mutation conferring resistance to an amiRNA targeted to said gene involved in pollen development is a conservative mutation.

68. The method of claim 62, wherein the gene involved in pollen development is a gene involved in pollen formation or pollen shedding; optionally
wherein the gene involved in pollen formation is from the MYB class of transcription factors; optionally
wherein the gene involved in pollen formation is MYB 103; optionally
wherein the gene involved in pollen formation is Arabidopsis thaliana MYB 103.

69. A male sterile transgenic plant produced by the method of claim 62.

70. Transgenic seed, propagating material or progeny of the transgenic plant of claim 69.

71. A method of producing male fertile hybrid progeny from a male sterile plant produced by a method comprising transforming the plant with an isolated nucleic acid encoding a precursor amiRNA targeted to a gene involved in pollen development; optionally
wherein the plant is transformed with a second isolated nucleic acid encoding a gene involved in pollen development said gene comprising at least one mutation conferring resistance to an amiRNA targeted to said gene involved in pollen development,
wherein said second nucleic acid is fused in frame to a ligand binding domain of the ecdysone receptor;
fertilizing the male sterile transgenic plant with pollen from a male fertility restorer transgenic plant produced by the method of claim 66; and
collecting the resulting male fertile hybrid seed.

72. Male fertile hybrid seed produced by the method of claim 71.

73. An isolated nucleic acid encoding a precursor amiRNA encoding an amiRNA targeted to a gene involved in pollen development; optionally
wherein the amiRNA contains a target sequence that is
(i) native to the plant being considered, or
(ii) heterologous to the plant being considered, or
(iii) synthetically generated, or
(iv) partially complementary to the target sequence, or
(v) fully complementary to the target sequence, or
(vi) fully complementary to the target sequence in the first ten to fifteen bases from the 5′ end of the amiRNA.

74. An isolated nucleic acid comprising a mutated copy of a gene involved in pollen development comprising at least one mutation conferring resistance to the amiRNA of claim 73; optionally
wherein the gene involved in pollen development is a gene involved in pollen formation or pollen shedding; or
wherein the gene involved in pollen development is
(i) a gene involved in pollen formation and is from the MYB class of transcription factors, or
(ii) a gene involved in pollen formation and is MYB103; or
(iii) a gene involved in pollen formation is Arabidopsis thaliana MYB 103.

75. The nucleic acid of claim 73, further comprising at least one promoter operably linked to the precursor amiRNA; optionally wherein the promoter is
(i) a native promoter, or
(ii) a heterologous promoter, or
(iii) an inducible promoter.

76. A vector or a host cell comprising the nucleic acid of claim 73.

77. A transgenic plant comprising the nucleic acid of claim 73.

78. Transgenic seed, propagating material or progeny of the transgenic plant of claim 77.

79. The method of claim 66, wherein the at least one mutation conferring resistance to an amiRNA targeted to said gene involved in pollen development is a conservative mutation.

80. A male, fertility restorer transgenic plant produced by the method of claim 66.

81. A male fertility restorer transgenic plant produced by the method of claim 67.

82. A method of producing male fertile hybrid progeny from a male sterile plant produced by a method comprising transforming the plant with an isolated nucleic acid encoding a precursor amiRNA targeted to a gene involved in pollen development; optionally
wherein the plant is transformed with a second isolated nucleic acid encoding a gene involved in pollen development said gene comprising at least one mutation conferring resistance to an amiRNA targeted to said gene involved in pollen development,
wherein said second nucleic acid is fused in frame to a ligand binding domain of the ecdysone receptor;
fertilizing the male sterile transgenic plant with pollen from a male fertility restorer transgenic plant produced by the method of claim 67; and
collecting the resulting male fertile hybrid seed.

83. A transgenic plant comprising the vector or the host cell of claim 76.


SEQUENCE LISTING (27 sequences)

SEQ ID NO: 1 - DNA, 176 bp, Arabidopsis thaliana
agagagagcttccttgagtccattcacaggtcgtgatatgattcaattagcttccgactcattcatccaaataccgagtcgccaaaattcaaactagactcgttaaatgaatgaatgatgcggtagacaaattggatcattgattctctttgattggactgaagggagctccctct

SEQ ID NO: 2 - DNA, 172 bp, Arabidopsis thaliana
agagagctttcttcggtccactcatggagtaatatgtgagatttaattgactctcgactcattcatccaaataccaaatgaaagaatttgttctcatatggtaaatgaatgaatgatgcgagagacaaattgagtcttcacttctctatgcttggactgaagggagctccct

SEQ ID NO: 3 - DNA, 199 bp, Arabidopsis thaliana
tagatatagaaggagattctttcagtccagtcatggatagaaaaagaagagggtagaaatatctgccgactcatccatccaaacactcgtggtagagaaacgataaatttaaaccgcagtgactgtgtgaatgatgcgggagatatttttgatccttctttatctgtgtttggactgaagggagctccttctttttcta

SEQ ID NO: 4 - DNA, 182 bp, Arabidopsis thaliana
tagagctccttaaagttcaaacatgagttgagcagggtaaagaaaagctgctaagctatggatcccataagccctaatccttgtaaagtaaaaaaggatttggttatatggattgcatatctcaggagctttaacttgccctttaatggcttttactcttctttggattgaagggagctcta

SEQ ID NO: 5 - DNA, 186 bp, Arabidopsis thaliana
aagagctccttgaagttcaatggagggtttagcagggtgaagtaaagctgctaagctatggatcccataagccttatcaaattcaatataattgatgataaggttttttttatggatgccatatctcaggagctttcacttacccctttaatggcttcactcttctttggattgaagggagctctt

SEQ ID NO: 6 - DNA, 225 bp, Arabidopsis thaliana
gtgtaacagaaggagctcccttcctccaaaacgaagaggacaagatttgaggaactaaaatgcagaatctaagagttcatgtcttcctcatagagagtgcgcggtgttaaaagcttgaagaaagcacactttaaggggattgcacgacctcttagattctccctctttctctacatatcattctcttctcttcgtttggattgaagggagctccttttcttcttc

SEQ ID NO: 7 - DNA, 32 bp, Artificial Sequence
  Primer 1 - BnF
cacaggatccgtgggggtcctgtaattgactc

SEQ ID NO: 8 - DNA, 83 bp, Artificial Sequence
  Primer 2 - miRNA R
cagcgagtattaagtgtatgaacatgtgtaatatgcgtccgagcgtgtgtttgtctttctttctagtttttatctctcgatcg

SEQ ID NO: 9 - DNA, 99 bp, Artificial Sequence
  Primer 3 - BnMu F
gttcatacacttaatactcgctgttttgaattgatgttttaggaatatatatgtagagcagagatgcttctcttgctttcacaggtcgtgatatgattc

SEQ ID NO: 10 - DNA, 43 bp, Artificial Sequence
  Primer 4 - BnMu R
gccgagatgcttcacttgctatcaaagagaatcaatgatccaa

SEQ ID NO: 11 - DNA, 49 bp, Artificial Sequence
  Primer 5 - BnMuFI
tagcaagtgaagcatctcggctctctcttttgtattccaattttcttga

SEQ ID NO: 12 - DNA, 35 bp, Artificial Sequence
  Primer 6 - miRNA P R
tgataagcttcatggcgatgccttaaataaagata

SEQ ID NO: 13 - DNA, 32 bp, Artificial Sequence
  Primer 7 - Mut Fori
cacaaagcttgtgggggtcctgtaattgactc

SEQ ID NO: 14 - DNA, 44 bp, Artificial Sequence
  Primer 8 - Mut Rev1
caagagatgcaacatttcatccttgaaacatccgagggcagctt

SEQ ID NO: 15 - DNA, 45 bp, Artificial Sequence
  Primer 9 - Mut For2
gatgaaatgttgcatctcttgaccaagaaacgtgttgatctaaac

SEQ ID NO: 16 - DNA, 35 bp, Artificial Sequence
  Primer 10 - Mut Rev2
ctgcgagctctcaaaccacatgattaatgagatca

SEQ ID NO: 17 - DNA, 46 bp, Artificial Sequence
  Primer 11 - 103MI R
atctctggcattagatggtagtctacatatatattcctaaaacatc

SEQ ID NO: 18 - DNA, 38 bp, Artificial Sequence
  Primer 12 - 103MI F
ctaccatctaatgccagagattcacaggtcgtgatatg

SEQ ID NO: 19 - DNA, 43 bp, Artificial Sequence
  Primer 13 - 103MUR
ctgccatctaatggcagagaatcaaagagaatcaatgatccaa

SEQ ID NO: 20 - DNA, 40 bp, Artificial Sequence
  Primer 14 - 103MUF1
ttctctgccattagatggcagtcttctttttgtattccaa

SEQ ID NO: 21 - DNA, 35 bp, Artificial Sequence; identical to SEQ ID NO: 12
  Primer 15 - miRNAPREV

SEQ ID NO: 22 - DNA, 32 bp, Artificial Sequence
  Primer 16 - MUTF1
cacaaagcttgtgggggtcctgtcattgactc

SEQ ID NO: 23 - DNA, 42 bp, Artificial Sequence
  Primer 17 - 103MR
atttcagccatcaagtgtgaaaaaggcttgtgggttacggga

SEQ ID NO: 24 - DNA, 42 bp, Artificial Sequence
  Primer 18 - 103MF
ttttcacacttgatggctgaaataaccactacactcaatcct

SEQ ID NO: 25 - DNA, 35 bp, Artificial Sequence; identical to SEQ ID NO: 16
  Primer 19 - MUTR2

SEQ ID NO: 26 - DNA, 29 bp, Artificial Sequence
  Primer 20 - 2MIF
aactagtcatggcgatgccttaaataaag

SEQ ID NO: 27 - DNA, 28 bp, Artificial Sequence
  Primer 21 - 2MIR
taagcttcaaacacacgctcggacgcat